(19) United States
(12) Patent Application Publication
(10) Pub. No.: US 2025/0320315 A1
(43) Pub. Date: Oct. 16, 2025

(54) BLETILLA STRIATA POLYSACCHARIDE, PREPARATION METHOD AND USE THEREOF, IMMUNE ADJUVANT AND NANO VACCINE CONTAINING THE SAME

(71) Applicants: KUNMING INSTITUTE OF BOTANY, CHINESE ACADEMY OF SCIENCES, Yunnan (CN); INSTITUTE OF MEDICAL BIOLOGY, CHINESE ACADEMY OF MEDICAL SCIENCES, Yunnan (CN)

(72) Inventors: Jiangmiao HU, Yunnan (CN); Ye LIU, Yunnan (CN); Liu YANG, Yunnan (CN); Sisi CHEN, Yunnan (CN); Jinyu LI, Yunnan (CN)

(21) Appl. No.: 17/423,635

(22) PCT Filed: May 12, 2021
(86) PCT No.: PCT/CN2021/093326

§ 371 Date: Jun. 5, 2025

(30) Foreign Application Priority Data
Mar. 27, 2021 (CN) 202110328789.3

(51) Int. Cl. C08B 37/00; A61K 31/715; A61K 36/898; A61K 39/00; A61K 39/215; A61P 37/02; B82Y 5/00; B82Y 40/00

(52) U.S. Cl. CPC C08B 37/006; A61K 31/715; A61K 36/898; A61K 39/215; A61P 37/02; C08B 37/0003; A61K 2039/55583; A61K 2236/10; A61K 2236/331; A61K 2236/51; A61K 2236/53; B82Y 5/00; B82Y 40/00

(57) ABSTRACT

Disclosed is a Bletilla striata polysaccharide, its preparation method and use, an immune adjuvant and a nano vaccine containing the Bletilla striata polysaccharide. The Bletilla striata polysaccharide of the disclosure has the immunomodulatory effect. The Bletilla striata polysaccharide and the protein vaccine are self-assembled to generate the nano vaccine after mixing, and the self-assembled nano vaccine is relatively uniform round particles. When the nano vaccine is used to immunize mice, it is able to activate antigen presenting cells (APCs) and improve their uptake of antigen. The nano vaccine enables mice to generate antibody and cell response which are 2-10 times stronger than that of the traditional vaccines.

### CROSS REFERENCE TO RELATED APPLICATION

The application claims the priority of Chinese Patent Application No. 202110328789.3 entitled “Bletilla striata polysaccharide, preparation method and use thereof, immune adjuvant and nano vaccine containing the same” filed on Mar. 27, 2021, the entire contents of which are incorporated herein by reference.

### TECHNICAL FIELD

The present disclosure relates to the field of immunomodulatory technology, in particular relates to a Bletilla striata polysaccharide, its preparation method and use, an immune adjuvant and a nano vaccine containing the Bletilla striata polysaccharide.

### BACKGROUND ART

The traditional Chinese medicine Bletilla striata is the dry tubers of the Bletilla striata (Thunb.) Reichb. f. of genus Bletilla in Orhidaceae. It has the effects of astringent hemostasis, detumescence and granulation-promoting, lung-tonifying and cough relief. Currently, the major chemical ingredients of the genus Bletilla reported in the literature are bibenzyls, phenanthrenes and derivatives thereof, steroidal saponins, triterpenoids, flavonoids, anthocyanins, and the like, which are more than 150 kinds. These ingredients exhibit a wide variety of biological activities in subsequent studies, such as anti-inflammatory, anti-oxidation, antibacterial, hemostasis, anti-tumor, and inhibition of tyrosinase.

Bletilla striata is rich in polysaccharides, and currently reported types of Bletilla striata polysaccharide skeletons are mainly 1,2- or 1,4-linked mannose residues and 1,4-linked glucose residues. The traditional preparation and use of Bletilla striata polysaccharide are focused on wound healing or interventional treatment of tumors as a processed plasma and vascular embolic agent.

### SUMMARY

An objective of the disclosure is to provide a Bletilla striata polysaccharide and its preparation method and use, an immune adjuvant and a nano vaccine containing the Bletilla striata polysaccharide. The Bletilla striata polysaccharide provided by the disclosure has the immunomodulatory effect.

In order to achieve the above objective, the following technical scheme is provided:

In a first aspect, the disclosure provides a Bletilla striata polysaccharide. The chemical structural formula of the Bletilla striata polysaccharide is shown in formula I. The relative molecular weight of the Bletilla striata polysaccharide is 42.0-42.5 KDa.

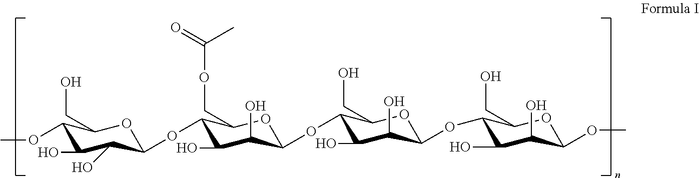

In some embodiments, a relative molecular weight of the Bletilla striata polysaccharide is 42.4 KDa.

In a second aspect, the disclosure provides a preparation method for the Bletilla striata polysaccharide, which includes the following steps:
1) degreasing Bletilla striata tuber powder to obtain a defatted raw material;
2) mixing the defatted raw material and water for water extraction to obtain a water extract;
3) precipitating the water extract with alcohol, separating solid from liquid, and collecting a precipitate;
4) deproteinizing the precipitate to obtain a Bletilla striata crude polysaccharide;
5) performing a first chromatographic column chromatography on the Bletilla striata crude polysaccharide sequentially by using water and an aqueous NaCl solution with a concentration of 0.05 mol/L, and collecting an elution part of the aqueous NaCl solution; performing a second chromatographic column chromatography on the elution part of the aqueous NaCl solution by using water, collecting an eluent, and detecting the eluent by an HPLC-ELSD; and collecting components with a peak time of 9.32 min to obtain the Bletilla striata polysaccharide.

In some embodiments, precipitating the water extract with alcohol in step 3) includes: filtering and concentrating the water extract sequentially to obtain a concentrated solution; mixing the concentrated solution and ethanol to obtain a mixture, and standing the mixture for alcohol precipitation. A volume concentration of ethanol in the mixture is more than 70%.

In some embodiments, deproteinization treatment in step 4) includes: redissolving the precipitate to obtain a redissolved solution; mixing the redissolved solution and a Sevage reagent in a volume ratio of 4:1, shaking and centrifuging the mixture to obtain a denatured protein located at the boundary between the redissolved solution and the Sevage reagent. The Sevage reagent is obtained by mixing chloroform and n-butanol in a volume ratio of 4:1.

In a third aspect, the disclosure provides a use of the Bletilla striata polysaccharide described in the above scheme or the Bletilla striata polysaccharide prepared by the preparation method described in the above scheme in the preparation of immunomodulatory products.

In some embodiments, the immunomodulatory products include immunomodulatory drugs, immunomodulatory cosmetics or immunomodulatory functional foods.

In a fourth aspect, the disclosure provides a use of the Bletilla striata polysaccharide described in the above scheme or the Bletilla striata polysaccharide prepared by the preparation method described in the above scheme as an immune adjuvant in the preparation of vaccines.

In a fifth aspect, the disclosure provides a nano vaccine, including a polysaccharide and a protein vaccine. The polysaccharide is the Bletilla striata polysaccharide described in the above scheme or the Bletilla striata polysaccharide prepared by the preparation method described in the above scheme.

In some embodiments, the protein vaccine includes a SARS-COV-2 RBD protein.

The disclosure provides a Bletilla striata polysaccharide. The Bletilla striata polysaccharide of the disclosure has an immunomodulatory effect. The Bletilla striata polysaccharide and the protein vaccine are self-assembled to generate a nano vaccine after mixing, and the self-assembled nano vaccine is relatively uniform round particles. When the nano vaccine is used to immunize mice, it is able to activate antigen presenting cells (APCs) and improve their uptake of antigen. The nano vaccine enables mice to generate antibody and cell response which are 2-10 times stronger than that of the traditional vaccines.

### BRIEF DESCRIPTION OF THE DRAWINGS

FIG. 1 is a flow chart shows the extraction of the Bletilla striata polysaccharide in Example 1.

FIG. 2 is a flow chart shows the separation and purification of the Bletilla striata polysaccharide in Example 1.

FIG. 3 is the HPLC chromatogram of the pure BPS product.

FIG. 4 is the PMP-HPLC chromatogram of the pure BPS product.

FIG. 5 is the IR spectrum of the pure BPS product.

FIG. 6 is the UV spectrum of the pure BPS product.

FIG. 7 shows all the hydrogen proton signals in the pure BPS product.

FIG. 8 shows all the hydrogen atom signals in the pure BPS product.

FIG. 9 shows the correlation between carbon and its attached hydrogen protons in the pure BPS product.

FIG. 10 shows the correlation between hydrogen protons and carbon (including aprotic carbon and two or three bonds away from protons) in the pure BPS product.

FIG. 11 shows the correlation between hydrogen protons and hydrogen protons in the pure BPS product.

FIG. 12 shows the correlation of carbon dimensions from protons to protons directly connected to carbon and proton carbon in the same proton spin system in pure BPS product.

FIG. 13 is an electron micrograph shows the self-assembly of the pure BPS product and SARS-COV-2 RBD protein, and the scale is 500 nm.

FIG. 14 shows the immunomodulatory activity of the nano vaccine formed by BPS and SARS-COV-2 RBD protein.

### DETAILED DESCRIPTION OF THE EMBODIMENTS

The present disclosure provides a Bletilla striata polysaccharide, and the chemical structural formula of which is shown as Formula I. The relative molecular weight of the Bletilla striata polysaccharide is in a range of 42.0-42.5 KDa;

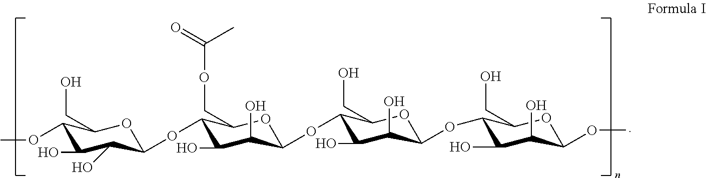

In the present disclosure, the relative molecular weight of the Bletilla striata polysaccharide is preferably 42.4 KDa.

In the present disclosure, the monosaccharide composition of the Bletilla striata polysaccharide is that mannose: glucose is 3.08:1. The repeating sequence of the Bletilla striata polysaccharide is the repeating unit of Formula I with 1,4-β-D-glucose (A), 1,4,6-β-D-mannose (C) and 1,4-β-D-mannose (B) as backbones, and the molar percentage content of the acetyl group in unit (C) is 7.54%.

The present disclosure further provides a preparation method for the Bletilla striata polysaccharide described in the above scheme, including the following steps:
1) degreasing Bletilla striata tuber powder to obtain a defatted raw material;
2) mixing the defatted raw material and water for water extraction to obtain a water extract;
3) precipitating the water extract with alcohol, separating solid from liquid, and collecting a precipitate;
4) deproteinizing the precipitate to obtain a Bletilla striata crude polysaccharide;
5) performing a first chromatographic column chromatography on the Bletilla striata crude polysaccharide sequentially by using water and an aqueous NaCl solution with a concentration of 0.05 mol/L, and collecting an elution part of the aqueous NaCl solution; performing a second chromatographic column chromatography on the elution part of the aqueous NaCl solution by using water, collecting an eluent, and detecting the eluent by an HPLC-ELSD; and collecting components with a peak time of 9.32 min to obtain the Bletilla striata polysaccharide.

In the present disclosure, firstly, the Bletilla striata tuber powder is degreased to obtain the defatted raw material.

In the present disclosure, the Bletilla striata tuber powder is preferably obtained by crushing Bletilla striata tuber. In the present disclosure, there is no particular limitation on the particle size of the Bletilla striata tuber powder, a conventional particle size in the field will do.

In the present disclosure, the step of degreasing treatment on the Bletilla striata tuber powder includes: mixing an aqueous ethanol solution with a volume concentration of 95% and the Bletilla striata tuber powder, and then leaching the same to obtain a leaching liquor; performing solid-liquid separation on the leaching liquor, and collecting precipitates to obtain the defatted raw material.

In the present disclosure, the number of leaching times is preferably 3 times. In the present disclosure, there is no particular limitation on the amount of the aqueous ethanol solution used for each leaching, a conventional amount in the art will do. In the present disclosure, the solid-liquid separation method for the leaching liquor is preferably filtration. The mesh size of the filter screen used for filtration is preferably 20-60 mesh, and more preferably 40 mesh.

In the present disclosure, after obtaining the defatted raw material, mixing and the defatted raw material and water, and extracting the mixture with water to obtain a water extract. In the present disclosure, the water preferably includes deionized water. The method of water extraction preferably includes reflux extraction. The temperature of the reflux extraction is preferably 80-100° C. The number of the reflux extraction is preferably 3 times, and the duration for each reflux extraction is preferably 1-3 h.

In the present disclosure, after obtaining the water extract, depositing the water extract with alcohol for solid-liquid separation, and collecting the precipitate. In the present disclosure, the alcohol precipitation method of the water extract by using ethanol includes: filtering and concentrating the water extract to obtain a concentrated solution; mixing the concentrated solution and ethanol to obtain a mixture, and standing the mixture for alcohol precipitation. A volume concentration of ethanol in the mixture is preferably more than 70%. In the present disclosure, the mesh size of the filter screen used for filtration is preferably 0.25-0.45 μm. In the present disclosure, there is no particular limitation on the concentration method, a conventional concentration method in the art will do. During the specific implementation of the present disclosure, the concentration is vacuum concentration. In the present disclosure, the solid-liquid separation is preferably centrifugation. The rotational speed of centrifugation is preferably 3000-5000 rpm, and the centrifugal time is preferably 20-25 min.

In the present disclosure, after obtaining the precipitate, deproteinizing the precipitate to obtain a Bletilla striata crude polysaccharide. During the specific implementation of the present disclosure, the method for deproteinating the precipitate is as follows: redissolving the precipitate to obtain a redissolved solution; according to the denaturation characteristic of protein in organic solvents such as chloroform, mixing the redissolved solution and a Sevage reagent in a volume ratio of 4:1, shaking and centrifuging the mixture, the denatured protein is located at the boundary between the extracting solution and the Sevage reagent. The Sevage reagent is obtained by mixing chloroform and n-butanol in a volume ratio of 4:1. The method to remove the protein in the supernatant by using the Sevage reagent is under mild conditions and will not cause the denaturation of polysaccharide. In the present disclosure, removing the protein from the supernatant is preferably conducted 3 times. In the present disclosure, the reagent used for redissolving the precipitate is preferably water. The temperature of the water is preferably 80-100° C. There is no particular restriction on the amount of the water, an amount sufficient to dissolve the precipitation will do.

In the present disclosure, after obtaining the Bletilla striata crude polysaccharides, performing a first chromatographic column chromatography on the Bletilla striata crude polysaccharide sequentially by using water and an aqueous NaCl solution with a concentration of 0.05 mol/L, and collecting an elution part of the aqueous NaCl solution; performing a second chromatographic column chromatography on the elution part of the aqueous NaCl solution by using water, collecting an eluent, and detecting the eluent by an HPLC-ELSD; and collecting components with a peak time of 9.32 min to obtain the Bletilla striata polysaccharide.

In the present disclosure, the chromatographic column used for the first chromatographic column chromatography is preferably DEAE-52 chromatographic column (80×4 cm).

In the present disclosure, the chromatographic column used for the second chromatographic column chromatography is preferably DEAE-Sepharose Fast Flow, Sephadex G-75 and Sephacryl S-200. During the specific implementation of the present disclosure, the elution part of the aqueous NaCl solution with a concentration of 0.05 mol/L is eluted by repeated column chromatography with DEAE-Sepharose Fast Flow, Sephadex G-75 and Sephacryl S-200.

In the present disclosure, the conditions of HPLC-ELSD detection include: chromatographic column: TSKgel-G4000Wx1 7.8*30; mobile phase: water; 100% isocratic elution; time: 0-15 min; and sample size: 10 mL.

The present disclosure further provides a use of the Bletilla striata polysaccharide described in the above scheme or the Bletilla striata polysaccharide prepared by the preparation method described in the above scheme in the preparation of immunomodulatory products. In the present disclosure, the immunomodulatory products preferably include immunomodulatory drugs, immunomodulatory cosmetics or immunomodulatory functional foods.

The present disclosure further provides a use of the Bletilla striata polysaccharide described in the above scheme or the Bletilla striata polysaccharide prepared by the preparation method described in the above scheme as an immune adjuvant in the preparation of vaccines.

The present disclosure further provides a nano vaccine, including a polysaccharide and a protein vaccine. The polysaccharide is the Bletilla striata polysaccharide described in the above scheme or the Bletilla striata polysaccharide prepared by the preparation method described in the above scheme. In the present disclosure, the mass ratio of the polysaccharide and the protein vaccine is preferably (4-50): (1-5), and further preferably (10-30): (2-3).

In the present disclosure, the protein vaccine preferably includes a SARS-COV-2 RBD protein.

The present disclosure further provides a preparation method for the nano vaccine described in the above scheme, including the following steps:

mixing the Bletilla striata polysaccharide and the protein vaccine to obtain a nano vaccine.

In the present disclosure, the mixing step includes: mixing the Bletilla striata polysaccharide and the protein vaccine with water respectively to obtain a Bletilla striata polysaccharide aqueous solution and a protein vaccine aqueous solution respectively. The concentration of the Bletilla striata polysaccharide in the Bletilla striata polysaccharide aqueous solution is preferably 2-5 mg/ml, more preferably 3-4 mg/ml. The concentration of the protein vaccine aqueous solution is preferably 1-5 mg/ml, more preferably 2-3 mg/ml. Before mixing, the protein vaccine aqueous solution is preferably subjected to a 0.25 μm filter membrane for filtration treatment. The volume ratio of the aqueous solution of the Bletilla striata polysaccharide to the aqueous solution of protein vaccine is preferably (2-10): 1, and more preferably (5-8):1.

In the present disclosure, the temperature of the mixing is preferably 20-30° C., more preferably 25° C. In the present disclosure, there is no particular limitation on the mixing time, whichever and the mixing is uniform.

The technical solutions of the present disclosure will be clearly and completely described below with reference to the examples of the present disclosure. Obviously, the described examples are only a part of the examples of the present disclosure, rather than all the examples. Based on the examples of the present disclosure, all other examples obtained by those skilled in the art without creative works shall fall within the protection scope of the present disclosure.

### Example 1: Extraction, Separation, Purification and Structure Identification of the Bletilla Striata Polysaccharide

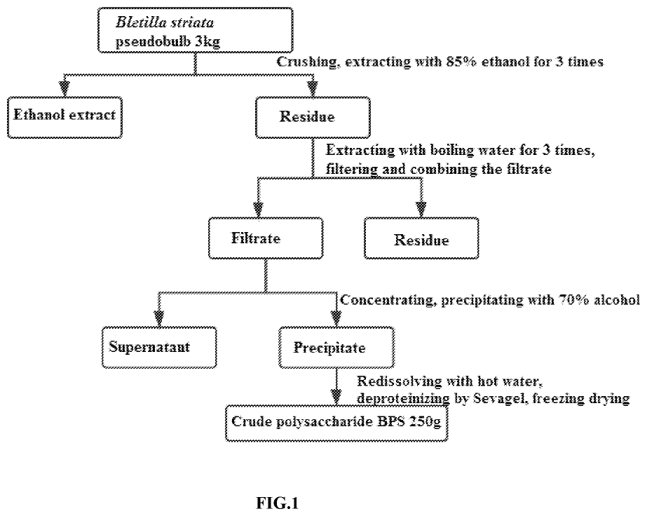

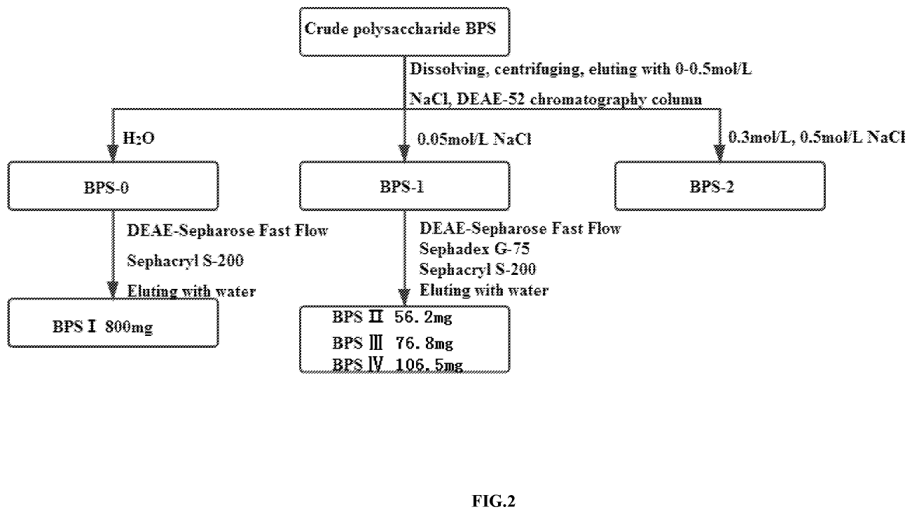

The flow chart of the extraction of the Bletilla striata polysaccharide is shown in FIG. 1. The flow chart of the separation and purification of the Bletilla striata polysaccharide is shown in FIG. 2.
1. 3 kg of dry Bletilla striata tuber powder was taken, 85% ethanol was added for leaching three times. The leaching liquor was filtered by filter paper with a mesh size of 0.25-0.45 μm. The filter residue was placed at room temperature, ventilated and dried, and then extracted with boiling water. 20 L of distilled water was added to the dried filter residue for extraction twice with boiling water for 2 h.
2. Solution after boiling water extraction was filtered, and the filtrate was combined and concentrated at 65° C. under reduced pressure to 10 L. 95% ethanol was added slowly to the concentrated solution, and stirred quickly to make the final alcohol concentration reach 70%, stood overnight, the concentrated solution was centrifuged after standing overnight (5000 rpm, 20 min). The precipitate obtained was redissolved with 10 L of hot water, and the redissolved solution was centrifuged to remove the filter residue.
3. The supernatant obtained by centrifugation and the Sevage reagent (obtained by mixing chloroform and n-butanol in a volume ratio of 4:1) were mixed in a volume ratio of 4:1, the mixture was shaken and centrifuged, the deformed protein was located at the boundary between the extracting solution and the Sevage reagent. The step 3 was repeated twice. The residue obtained was concentrated and freeze-dried, 250 g of Bletilla striata crude polysaccharide BPS was obtained.
4. 400 mg of BPS was taken each time, 3 mL of water was added for heating dissolution. The solution was cooled at room temperature, and centrifuged at a speed of 3000 rpm for 5 min to obtain a supernatant. The supernatant was eluted with a distilled water, 0.05 mol/L, 0.3 mol/L, and 0.5 mol/L aqueous NaCl solution in sequence with DEAE-52 chromatography column (80×4 cm). Each component was collected by an automatic sampler. The components with the same peak time was detected by HPLC-ELSD, and the components combined and dialyzed. Each component was concentrated under reduced pressure and freeze-dried in vacuum, three components were obtained: BPS-0 (elution part of water), BPS-1 (elution part of 0.05 mol/L aqueous NaCl solution) and BPS-2 (elution part of 0.3 mol/L and 0.05 mol/L aqueous NaCl solution; because of low content and deep color, the elution part did not continue to be purified). BPS-0 was further eluted by repeated column chromatography with DEAE Sepharose Fast Flow and Sephacryl S-200, the chromatographic columns were eluted with water, the eluent was collected by an automatic sample receiver, every other tube of samples was detected by HPLC-ELSD and combined to obtained BPS I. BPS-1 was further eluted by repeated column chromatography with DEAE Sepharose Fast Flow, Sephadex G-75 and Sephacryl S-200, the chromatographic columns were eluted with water, the eluent was collected by an automatic sample receiver, every two tubes of samples was detected by HPLC-ELSD (The conditions of HPLC-ELSD were as follows: chromatographic column: TSKgel-G4000Wx1 7.8*30; mobile phase: water; 100% isocratic elution; time: 0-15 min; and sample size: 10 mL) and combined, the combined solution was separated and purified to obtain BPS II, BPS III and BPS IV. BPS II is the target product, i.e. the Bletilla striata polysaccharide with immunomodulatory effects.

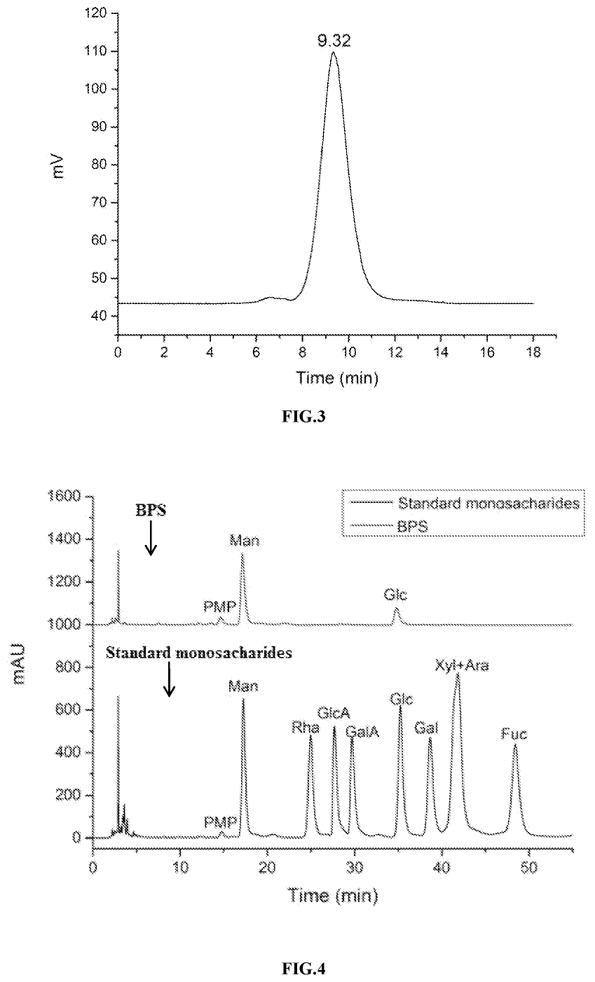

The HPLC chromatogram of the pure BPS product (BPS II) is shown in FIG. 3. The homogeneous polysaccharide was analyzed by methylation analysis of polysaccharide link component (PMP-HPLC), infrared spectrum analysis, ultraviolet spectrum analysis and optical rotation analysis, and NMR spectrometry, and the finally determined structure of the homogeneous polysaccharide was: relative molecular weight was approximately equal to 42.4 KDa; the monosaccharide composition was mannose: glucose is 3.08:1. The polysaccharide repeated sequence of is the repeating unit as shown in the figure below with 1,4-β-D-glucose (A), 1,4,6-β-D-mannose (C) and 1,4-β-D-mannose (B) as backbones, and the content of acetyl group on the unit C was 7.54%, and the structure is as follows:

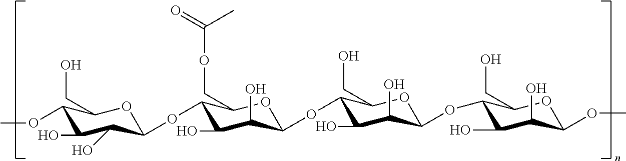

The results of the methylation analysis of polysaccharide link component are shown in FIG. 4.

The results of infrared (IR) spectrum analysis are shown in FIG. 5.

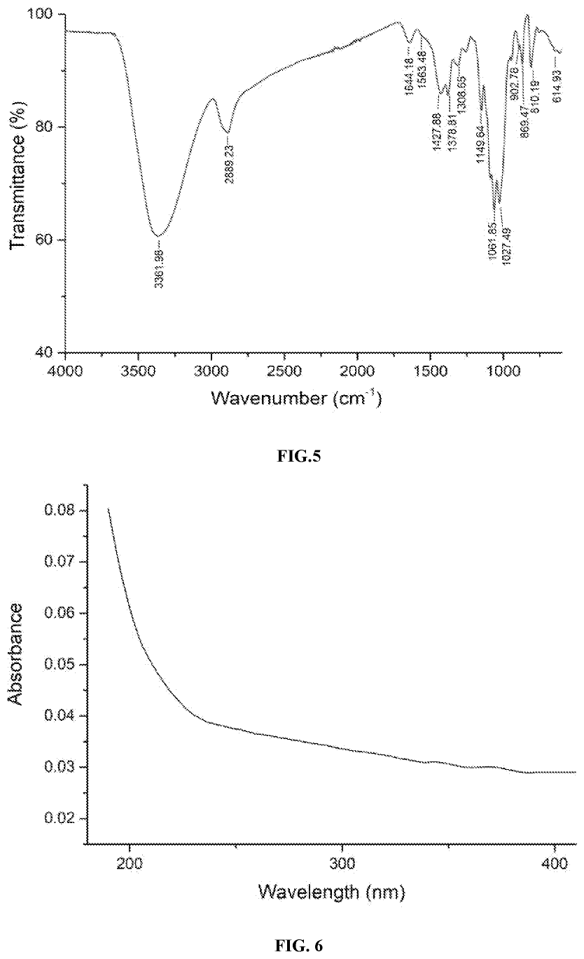

The results of ultraviolet and optical rotation analysis are shown in FIG. 6.

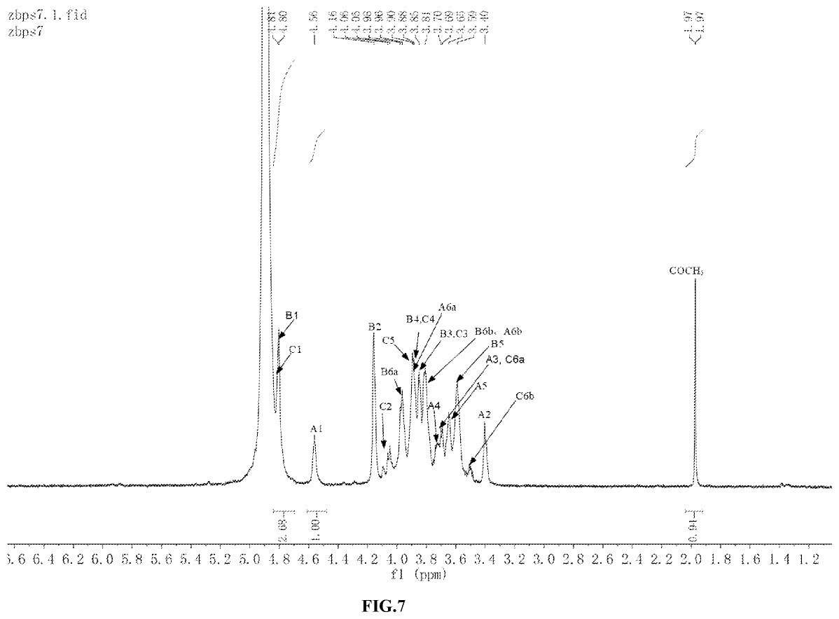

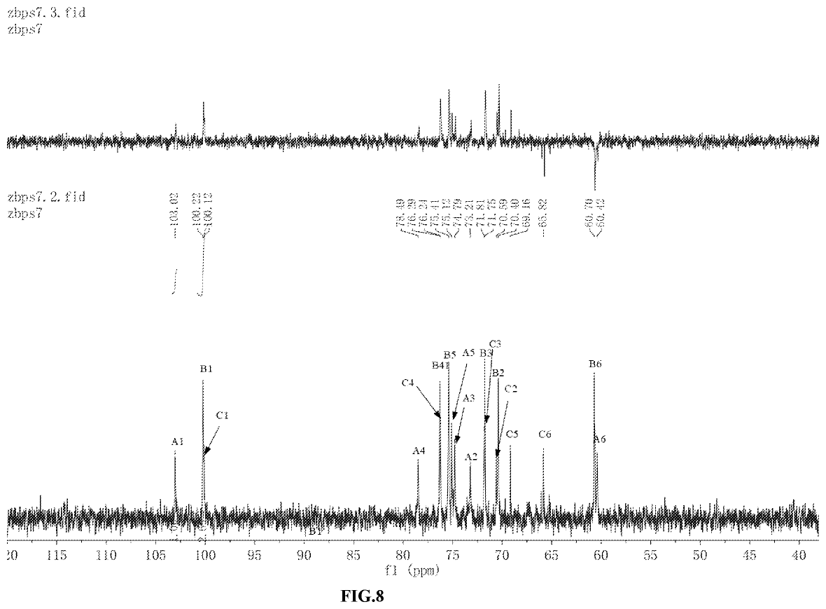

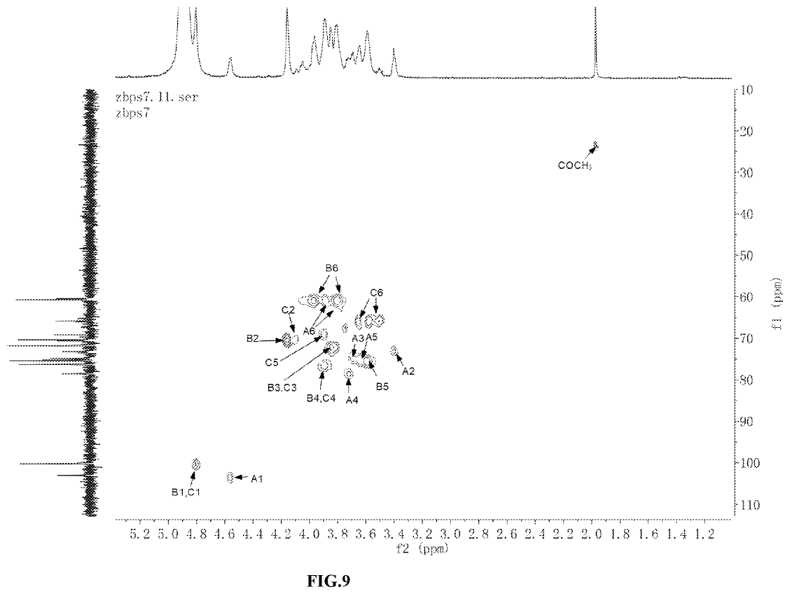

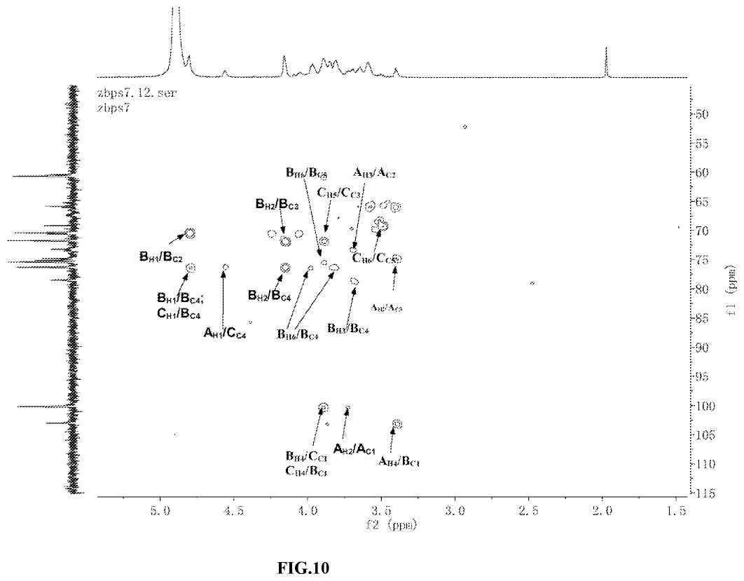

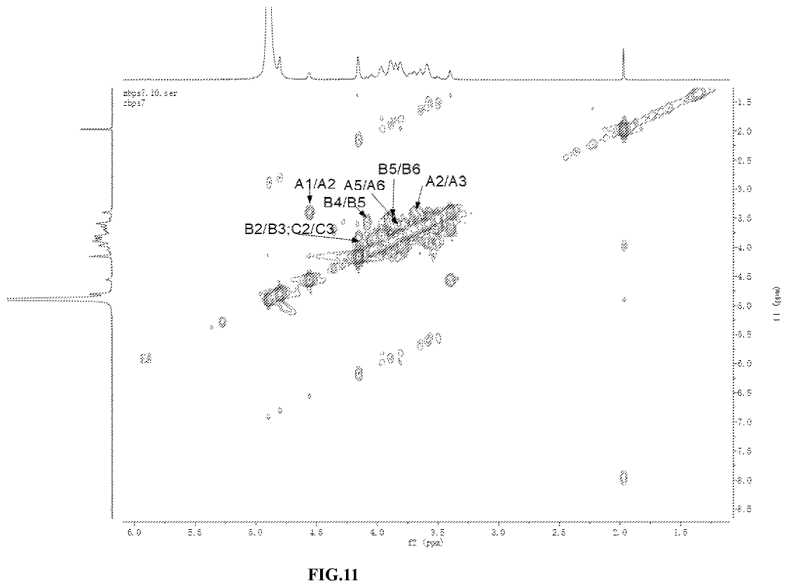

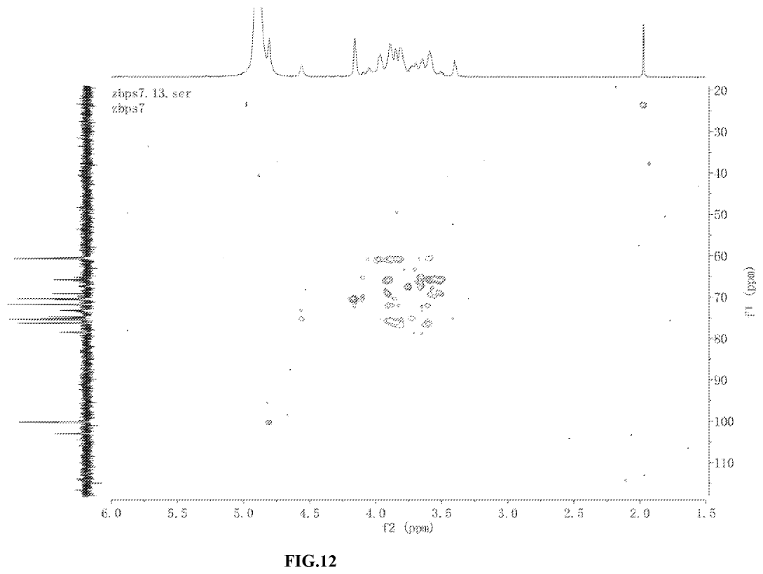

NMR spectrometry is an important means for determining the structural composition of the pure BPS product and the monosaccharide linkage. The results of NMR spectrometry are shown in FIG. 7 to FIG. 12. FIG. 7 shows all the hydrogen proton signals in the pure BPS product. FIG. 8 shows all the hydrogen atom signals in the pure BPS product. FIG. 9 shows the correlation between carbon and its attached hydrogen protons in the pure BPS product. FIG. 10 shows the correlation between hydrogen protons and carbon (including aprotic carbon and two or three bonds away from protons) in the pure BPS product, and the structure has heteronuclear multi bond correlation. FIG. 11 shows the correlation between hydrogen protons and hydrogen protons in the pure BPS product. FIG. 12 shows the correlation of carbon dimensions from protons to protons directly connected to carbon and proton carbon in the same proton spin system in pure BPS product.

### Example 2: Preparation of Nano Vaccine

10 mg of the Bletilla striata homogeneous polysaccharide was dissolved in 2 mL of pure water to configure an aqueous solution of polysaccharide at a concentration of 5 mg/mL, and the aqueous solution of polysaccharide was filtered through a 0.25 μm membrane for use. SARS-COV-2 RBD protein was mixed with pure water to prepare an aqueous solution of protein vaccine at a concentration of 3 mg/mL, and the aqueous solution of protein vaccine was filtrated through a 0.25 μm filtration membrane for use. In the pure aqueous buffer (pH 7.0), the prepared aqueous solution of polysaccharide and the aqueous solution of SARS-COV-2 RBD protein vaccine were mixed uniformly in a ratio of 5:1 to form a nano vaccine, which was ready for use.

### Example 3: Transmission Electron Microscope of Nano Vaccine

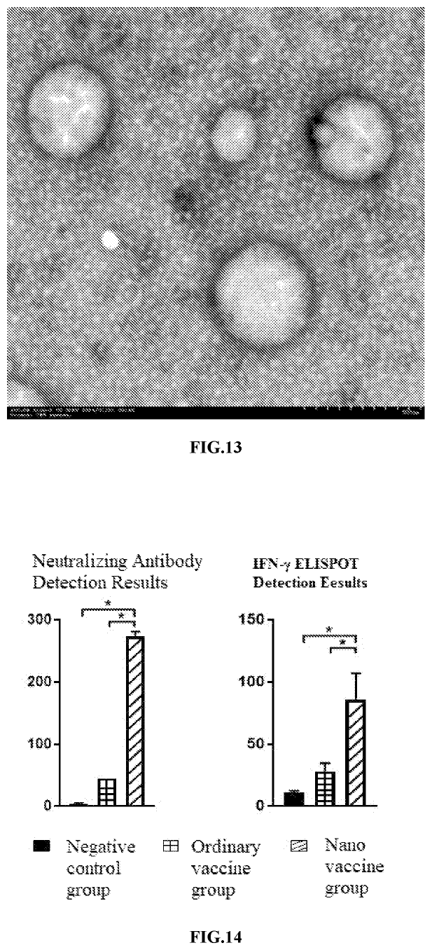

10 μL of the nano vaccine prepared in Example 2 above was taken, and the morphology of the nano vaccine was observed with a transmission electron microscope (TEM). The results are shown in FIG. 13, and it can be seen from FIG. 13 that the homogeneous polysaccharide and the vaccine are self-assembled into uniform circular particles, that is, the nano vaccine.

### Example 4: Immunomodulatory Activity of Nano Vaccine Formed by Polysaccharide BPS and SARS-COV-2 RBD Protein

Specific method for animal immunization: The nano vaccines prepared in Example 2 was used to immunize the animals. The animals were SPF BALB/c mice, 6 to 8 weeks old, which were fed in a sterile environment, and were divided into 3 groups with 5 mice in each group. Specific animal experiments were grouped as follows:

Negative control group: intramuscular injection of saline 100 μL/mouse;

Ordinary vaccine group: intramuscular injection of SARS-COV-2 RBD protein vaccine solution 10 μg/mouse;

Nano vaccine group: intramuscular injection of BPS-RBD nano vaccine 10 μg/mouse;

The immunization time intervals of the above three groups of animals were 0, 2 and 4 weeks, and ten days after the last immunization, blood samples were collected for pseudovirus neutralization assay and IFN-γ ELISPOT detection. The test results are shown in Table 1, Table 2 and FIG. 14, and it can be seen from Table 1, Table 2 and FIG. 14 that the activity of the assembled nano vaccine is significantly different from that of the ordinary vaccine.

TABLE 1
Neutralizing Antibody Detection Results

| Group | Neutralizing antibody detection |
|---|---|
| Negative control group | 3.5 |
| Ordinary vaccine group | 45.2 |
| Nano vaccine group | 279 |

TABLE 2
IFN- γ ELISPOT detection results

| Group | IFN-γ ELISPOT detection |
|---|---|
| Negative control group | 11 |
| Ordinary vaccine group | 28 |
| Nano vaccine group | 86 |

All animal experiments were approved by the Animal Experiment Ethics Committee of Institute of Medical Biology, Chinese Academy of Medical Sciences, and operated in strict accordance with the Regulations on the administration of laboratory animals in Yunnan Province and Ethics Committee Regulation.

The above are only the preferred embodiments of the disclosure. It should be pointed out that for ordinary skill in the technical field, a number of improvements and refinements can be made without departing from the principle of the disclosure, and these improvements and refinements should also be regarded as the protection scope of the disclosure.

What is claimed is:

1. A Bletilla striata polysaccharide, wherein the structural formula of the Bletilla striata polysaccharide is as shown in Formula I; and a relative molecular weight of the Bletilla striata polysaccharide is 42.0-42.5 KDa;

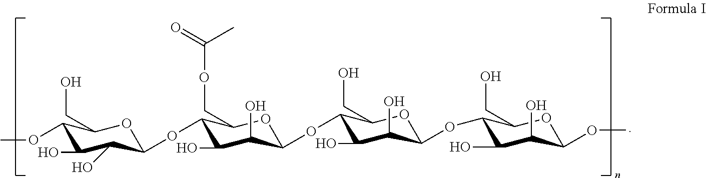

2. The Bletilla striata polysaccharide of claim 1, wherein a relative molecular weight of the Bletilla striata polysaccharide is 42.4 KDa.

3. A preparation method for the Bletilla striata polysaccharide of claim 1 or 2, comprising the following steps:
1) degreasing Bletilla striata tuber powder to obtain a defatted raw material;
2) mixing the defatted raw material and water for water extraction to obtain a water extract;
3) precipitating the water extract with alcohol, separating solid from liquid, and collecting a precipitate;
4) deproteinizing the precipitate to obtain a Bletilla striata crude polysaccharide;
5) performing a first chromatographic column chromatography on the Bletilla striata crude polysaccharide sequentially by using water and an aqueous NaCl solution with a concentration of 0.05 mol/L, and collecting an elution part of the aqueous NaCl solution; performing a second chromatographic column chromatography on the elution part of the aqueous NaCl solution by using water, collecting an eluent, and detecting the eluent by an HPLC-ELSD; and collecting components with a peak time of 9.32 min to obtain the Bletilla striata polysaccharide.

4. The preparation method of claim 3, wherein precipitating the water extract with ethanol in step 3) further comprises:
filtering and concentrating the water extract sequentially to obtain a concentrated solution;
mixing the concentrated solution and the ethanol to obtain a mixture; and
standing the mixture for alcohol precipitation; and a volume concentration of ethanol in the mixture is more than 70%.

5. The preparation method of claim 3, wherein deproteinizing the precipitate in step 4) further comprises:
redissolving the precipitate to obtain a redissolved solution;
mixing the redissolved solution and a Sevage reagent in a volume ratio of 4:1, shaking and centrifuging the mixture; a denatured protein is located at a boundary between the redissolved solution and the Sevage reagent; and the Sevage reagent is obtained by mixing chloroform and n-butanol in a volume ratio of 4:1.

6. Use of the Bletilla striata polysaccharide of claim 1 or 2 or the Bletilla striata polysaccharide prepared by the preparation method of claim 3 or 4 in the preparation of immunomodulatory products.

7. The use of claim 6, wherein the immunomodulatory products comprise immunomodulatory drugs, immunomodulatory cosmetics or immunomodulatory functional foods.

8. Use of the Bletilla striata polysaccharide of claim 1 or 2 or the Bletilla striata polysaccharide prepared by the preparation method of claim 3 or 4 as an immune adjuvant in the preparation of vaccines.

9. A nano vaccine, comprising a polysaccharide and a protein vaccine, wherein the polysaccharide is the Bletilla striata polysaccharide of claim 1 or 2 or the Bletilla striata polysaccharide prepared by the preparation method of claim 3 or 4.

10. The nano vaccine of claim 9, wherein the protein vaccine comprises a SASH-COV-2 RBD protein.
